(19) United States
(12) Patent Application Publication
(10) Pub. No.: US 2021/0113804 A1
(43) Pub. Date: Apr. 22, 2021

(54) Apparatuses and Methods for Anesthetizing a Dental Patient

(71) Applicant: Nicholas John Pelachyk, Clinton, MI (US)

(72) Inventor: Nicholas John Pelachyk, Clinton, MI (US)

(21) Appl. No.: 17/073,302

(22) Filed: Oct. 17, 2020

Related U.S. Application Data
(60) Provisional application No. 62/923,569, filed on Oct. 20, 2019.

(51) Int. Cl. A61M 19/00; A61C 5/82; A61M 39/10; A61M 5/145; A61C 19/06; A61M 5/158

(52) U.S. Cl. CPC A61M 19/00; A61C 5/82; A61M 39/10; A61M 5/1452; A61M 2005/1581; A61M 2210/02; A61M 2039/1038; A61M 2210/0625; A61C 19/063

(57) ABSTRACT

Apparatuses and methods are disclosed for anesthetizing a dental patient during dental procedures such as a root canal. Administering an anesthetic directly into the jaw bone of a patient, called intraosseous injection, is highly effective but can have a relatively short duration. In addition, a dental dam is often placed over the tooth being treated in order to control the environment. The apparatuses and methods disclosed herein permit an anesthetic to be delivered multiple times to the dental patient without removing or disturbing the dental dam.

### CROSS REFERENCE TO RELATED APPLICATION

This application claims the benefit of provisional application Ser. No. 62/923,569, filed Oct. 20, 2019, the disclosure of which is incorporated herein by reference.

### TECHNICAL FIELD

The present disclosure generally relates to apparatuses and methods for anesthetizing a dental patient during dental procedures such as a root canal. Oftentimes it is necessary to administer an anesthetic to the tooth being treated for the comfort of the dental patient. Delivering the anesthetic directly into the jaw bone of a patient, called intraosseous injection, is quick and highly effective but can have a relatively short duration. In addition, a dental dam is often placed over the tooth being treated in order to control the environment. The apparatuses and methods disclosed herein permit intraosseous injection of an anesthetic into the jaw bone of the dental patient multiple times without removing or disturbing the dental dam.

### BACKGROUND

As background, dental providers such as dentists and endodontists typically administer a local anesthetic, such as articaine or lidocaine, to dental patients for the duration of most dental procedures. The local anesthetic numbs the tooth subject to the dental procedure and permits the dental patient to have a relatively pain-free experience. The local anesthetic may be delivered to the dental patient in several ways, including but not limited to inferior alveolar nerve block (IANB), buccal infiltration, and intraosseous injection. The first two techniques primarily inject the anesthetic into the soft tissue around the tooth, and they have the advantage that they are relatively easy to administer and a single injection is effective for most patients. However, there are several disadvantages to IANB and buccal infiltration. First, the anesthetic can require a relatively long time to take effect, and, in some patients, a second injection must be given to fully numb the patient. Second, the duration of the anesthetic's effect can be somewhat short, requiring the anesthetic to be administered a second or third time during the procedure. Finally, these techniques may also have a lower success rate on some patients, which may require a supplemental injection of anesthetic to fully numb the patient.

The intraosseous injection technique injects the anesthetic directly into the jaw bone of the dental patient. This technique has the advantage that it may take effect very quickly and may generally have a higher rate of success over a wide range of patients having various dental maladies. However, administering the anesthetic in this manner is more complicated, as the provider must drill a hole into the patient's jaw bone in order to gain access to the cancellous portion of the jaw bone. This is typically accomplished using a hollow drill bit which leaves behind a cannula or sleeve embedded into the jaw bone through which the anesthetic may be delivered. As an example, the X-Tip® Drill and Guide Sleeve from Dentsply® Maillefer® (Ballaigues, Switzerland) may be used to drill a hole in the jaw bone of the patient in order to anesthetize dental patients via intraosseous injection. As another example, the Stabident system from Fairfax Dental, Inc., (Miami, Fla.) may also be used to gain access to the cancellous portion of the jaw bone. However, for this method the duration of the anesthetic's effect may be relatively short; and in some instances, the provider may need to administer the anesthetic a second time or third time in order to maintain the comfort of the patient.

Furthermore, for some dental procedures, the provider may place a “dental dam” over the tooth (or teeth) subject to the procedure. The dental dam may isolate the tooth from the rest of the mouth and may prevent the patient's saliva from interfering with the dental procedure, which could potentially contaminate the tooth with microorganisms, possibly leading to a post-procedural infection. In addition, the dental dam may prevent dental instruments and/or other dental materials from coming into contact with the patient, which may cause discomfort to the patient or may increase the risk of the patient accidentally swallowing such items. The dental dam usually blankets the tooth in a manner which causes the injection site for the anesthetic to be covered up. The dental dam may be constructed of latex or nitrile, and it may be installed by the provider at the beginning of the dental procedure.

In cases where a dental dam is used and a re-administration of the anesthetic to the patient is necessary, the provider may be required locate the original intraosseous injection site underneath the dental dam. Depending on the location of the injection site in relation to objects such as the dental dam, clamps, and so forth, access may be quite challenging. Temporary removal of the dental dam may at times be the best option to properly access the intraosseous injection site. This can cause frustration for the provider, discomfort for the patient, and will likely extend the procedure time.

Accordingly, a solution is needed for dental procedures which use a dental dam, and which require multiple administrations of the anesthetic. The apparatuses and methods disclosed herein overcome the potential inherent time duration disadvantage in intraosseous injection for dental procedures using a dental dam by allowing the provider to deliver the anesthetic multiple times to the patient without the provider having to remove or disturb the dental dam.

Limitations in the prior art will become apparent to those skilled in the art after reviewing the specification below and the corresponding drawings.

### SUMMARY

In one embodiment, an apparatus for delivering an anesthetic into a jaw bone of a dental patient is disclosed. The apparatus comprises a flexible tube having a first end and a second end, a needle, and a coupler, wherein: the first end of the flexible tube is configured to be fluidly coupled to a delivery system capable of injecting the anesthetic into the flexible tube; the second end of the flexible tube is fluidly coupled to the needle such that the needle is at substantially a right angle to the second end of the flexible tube; the needle is configured to be inserted into a proximal end of a cannula and, when inserted, is capable of delivering the anesthetic from the flexible tube into the cannula; the coupler is disposed at the second end of the flexible tube, surrounds a portion of the needle, and is configured to be removably affixed to the proximal end of the cannula, wherein: when the coupler is affixed to the proximal end of the cannula, the coupler is configured to fluidly seal the second end of the flexible tube to the cannula; and when a distal end of the cannula is inserted into the jaw bone of the dental patient, the needle is inserted into the proximal end of the cannula, the coupler is affixed to the proximal end of the cannula, and the delivery system is fluidly coupled to the first end of the flexible tube, the delivery system is capable of delivering the anesthetic through the flexible tube, the needle, and the cannula into the jaw bone of the dental patient one or more times.

In another embodiment, an apparatus for delivering an anesthetic into a jaw bone of a dental patient is disclosed. This apparatus comprises a delivery system, a flexible tube having a first end and a second end, a needle, a coupler, and a cannula having a proximal end and a distal end, wherein: the first end of the flexible tube is configured to be fluidly coupled to the delivery system capable of injecting the anesthetic into the flexible tube; the second end of the flexible tube is fluidly coupled to the needle such that the needle is at substantially a right angle to the second end of the flexible tube; the needle is configured to be inserted into the proximal end of the cannula and, when inserted, is capable of delivering anesthetic from the flexible tube into the cannula; the coupler is disposed at the second end of the flexible tube, surrounds a portion of the needle, and is configured to be removably affixed to the proximal end of the cannula, wherein: when the coupler is affixed to the proximal end of the cannula, the coupler is configured to fluidly seal the second end of the flexible tube to the cannula; and when the distal end of the cannula is inserted into the jaw bone of the dental patient, the needle is inserted into the proximal end of the cannula, the coupler is affixed to the proximal end of the cannula, and the delivery system is fluidly coupled to the first end of the flexible tube, the delivery system is capable of delivering the anesthetic through the flexible tube, the needle, and the cannula into the jaw bone of the dental patient one or more times.

In yet another embodiment, a method is disclosed for delivering an anesthetic into a jaw bone of a dental patient. The method comprises: fluidly coupling a delivery system to a first end of a flexible tube; inserting a distal end of a cannula into the jaw bone of the dental patient in a manner that allows the dental patient to be anesthetized; fluidly coupling a second end of a flexible tube onto a proximal end of the cannula, wherein the second end of the flexible tube is configured to be at a substantially right angle to the cannula; and delivering the anesthetic from the delivery system through the flexible tube, the needle, and the cannula, and into the jaw bone of the dental patient one or more times.

### BRIEF DESCRIPTION OF THE DRAWINGS

The embodiments set forth in the drawings are illustrative and exemplary in nature and not intended to limit the inventions defined by the claims. The following detailed description of the illustrative embodiments can be understood when read in conjunction with the following drawings, where like structure is indicated with like reference characters and in which:

FIG. 1 depicts an apparatus for delivering an anesthetic into the jaw bone of a dental patient according to one or more embodiments shown and described herein;

FIG. 2 illustrates a flexible tube, a needle, a coupler, and cannula according to one or more embodiments shown and described herein;

FIGS. 3A and 3B show a flexible tube, a needle, a coupler, and cannula according to one or more embodiments shown and described herein;

FIG. 4 depicts a flexible tube, a needle, and a coupler according to one or more embodiments shown and described herein;

FIG. 5 illustrates an apparatus for delivering an anesthetic into the jaw bone of a dental patient according to one or more embodiments shown and described herein;

FIG. 6 shows a jaw bone, a cheek, a cannula, a flexible tube, and a coupler according to one or more embodiments shown and described herein;

FIG. 7 depicts a tooth, a jaw bone, and a cannula according to one or more embodiments shown and described herein;

FIG. 8 illustrates an apparatus for delivering an anesthetic into the jaw bone of a dental patient according to one or more embodiments shown and described herein; and

FIG. 9 shows a dental dam and flexible tube according to one or more embodiments shown and described herein.

### DETAILED DESCRIPTION

As used herein, the word “provider” is defined to be dentists, dental assistants, nurses, nurse practitioners, endodontists, and so forth. Although “provider” may indicate a single individual, it is also intended to signify two or more people acting in concert or one or more people acting under the direction of another. Also, as used herein, the phrase “jaw bone” is defined to be either the lower jaw bone (often referred to as the “jawbone,” “mandible,” or “lower jaw”) or the upper jaw bone (often referred to as the “maxilla”).

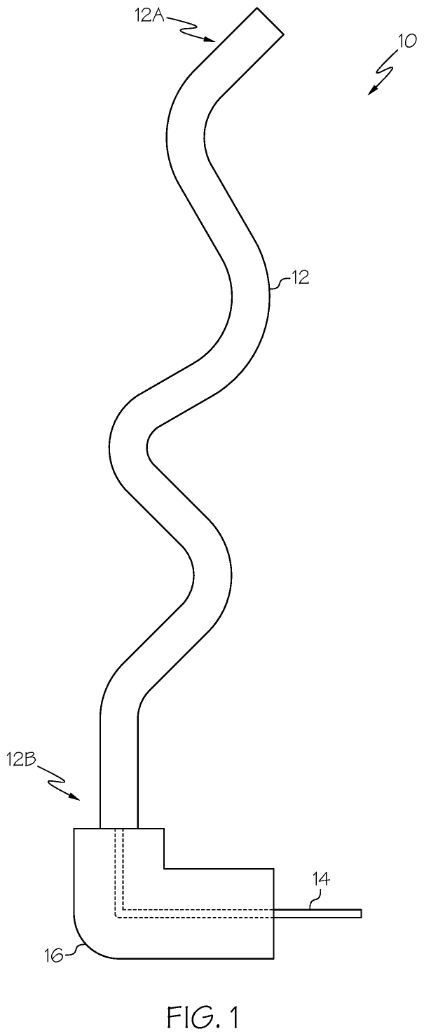

FIG. 1 depicts an apparatus 10 for delivering an anesthetic into the jaw bone of a dental patient according to one embodiment shown and described herein. The apparatus 10 may comprise a flexible tube 12 having a first end 12A and a second end 12B, a needle 14, and a coupler 16. The first end 12A of the flexible tube 12 may be configured to be fluidly coupled to a delivery system for the anesthetic. The delivery system may inject the anesthetic into the flexible tube 12 which is ultimately delivered into the jaw bone of the dental patient through a cannula. The second end 12B of the flexible tube 12 may be fluidly coupled to the needle 14, such that the needle 14 is at substantially a right angle with respect to the second end 12B of the flexible tube 12. That is, the longitudinal axis of the needle 14, as it protrudes from the coupler 16, may be at substantially a right angle with respect to the longitudinal axis of the second end 12B of the flexible tube 12.

The flexible tube 12 may be constructed of any suitable material capable of delivering anesthetic to the dental patient. For example, nylon may be used to construct the flexible tube 12. Also, the flexible tube 12 may of sufficient length to allow the delivery system for anesthetic to be place at a convenient location near the dental patient. Also, the flexible tube 12 may be clear or opaque in order to permit the provider to observe whether the anesthetic is present therein.

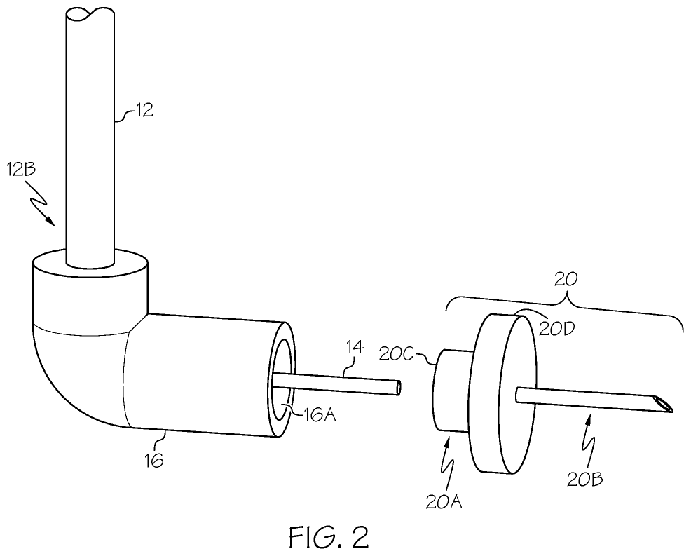

FIG. 2 illustrates an isometric view of the second end 12B of the flexible tube 12, the needle 14, the coupler 16, and the cannula 20. The needle 14 may be configured to be fluidly coupled to the second end 12B of the flexible tube 12. The needle 14 may be constructed of stainless steel or other suitable material. The cannula 20 may comprise a proximal end 20A, a distal end 20B, a male friction lock 20C, and a flange 20D. The needle 14 may be configured to be inserted into the proximal end 20A of the cannula 20. When the needle 14 is inserted into the proximal end 20A of the cannula 20, the needle 14 may deliver the anesthetic from the flexible tube 12 into the cannula 20. The diameter of the needle 14 may be such that it fits into the proximal end 20A of the cannula 20. That is, needle 14 and the cannula 20 may be concentric, and anesthetic delivered from the flexible tube 12 through the needle 14 may be directed into the hollow proximal end 20A of the cannula 20. A portion of the needle 14 may be surrounded by the coupler 16 as shown, and the needle 14 may have a bend in order to allow it to be at substantially a right angle with respect to the second end 12B of the flexible tube 12. The coupler 16 may have a female friction lock 16A which is designed to be removably affixed to the male friction lock 20C of the cannula 20. The flange 20D may provide a location mechanism that may prevent the distal end 20B of the cannula 20 from being inserted too far into the jaw bone of the patient. The flange 20D may be disposed at a fixed distance from the end of the distal end 20B that is based on the size of the patient and/or the location of the injection site.

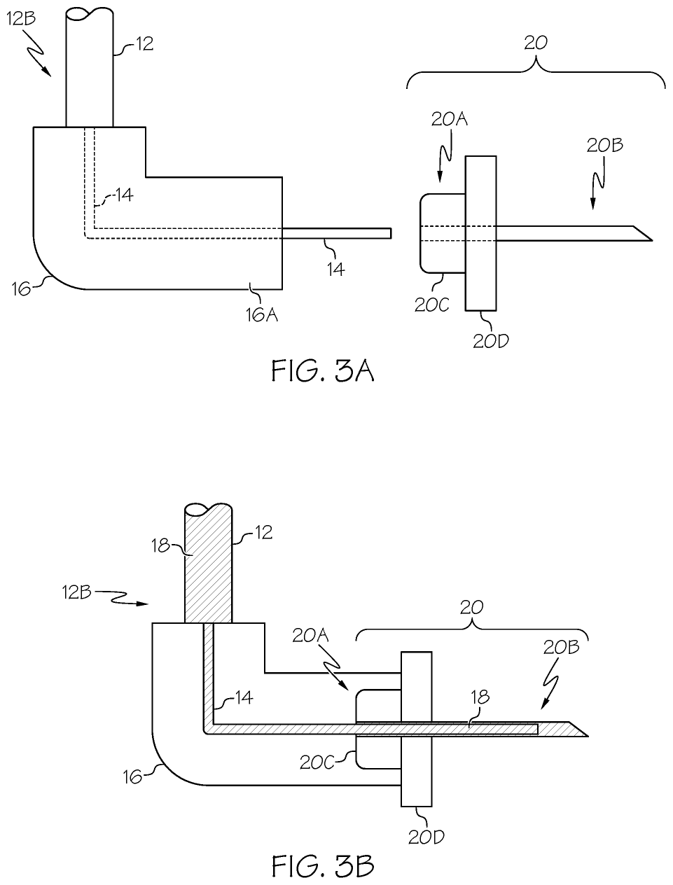

FIGS. 3A and 3B depict a side view of the second end 12B of the flexible tube 12, the needle 14, the coupler 16, and the cannula 20. In FIG. 3A, the coupler 16 is not affixed to the cannula 20. The cannula 20 may comprise a proximal end 20A and a distal end 20B, the general shape of which may be tubular. The proximal end 20A may be configured to be affixed to the coupler 16, and the distal end 20B may be configured to be inserted into the cancellous portion of the jaw bone of the dental patient. The coupler 16 may be have a female friction lock 16A which may be configured to be compatible with the male friction lock 20C of the cannula 20. The tip of the distal end 20B may be pointed in order to facilitate the insertion of the cannula 20 into the patient's jaw bone.

In FIG. 3B, the coupler 16 is affixed to the cannula 20, such that the needle 14 is inserted into the proximal end 20A of the cannula 20 and the female friction lock 16A of the coupler 16 is affixed to the male friction lock 20C of the cannula 20. In this case, the coupler 16 may form a fluid seal between the second end 12B of the flexible tube 12 and the proximal end 20A of the cannula 20. Also, when the coupler 16 is affixed to the cannula 20, there may be sufficient static friction between the male friction lock 20C and the female friction lock 16A to hold the coupler 16 affixed to the cannula 20 in spite of any force caused by the fluid pressure of the anesthetic 18 being delivered to the dental patient. However, the static friction may be low enough so that a dental provider may remove the coupler 16 from the cannula 20 with relative ease. As such, the coupler 16 may be removably affixed to the proximal end 20A of the cannula 20. Other coupling systems may be used in place of the female friction lock 16A and the male friction lock 20C, as is known in the art. For example, a Luer lock may be used to fluidly couple the flexible tube 12 to the cannula 20.

The distal end 20B of the cannula 20 may be inserted into the jaw bone of the dental patient such that the anesthetic 18 is capable of being delivered to the dental patient through the flexible tube 12, needle 14, and cannula 20. The second end 12B of the flexible tube 12 may be configured in a manner that it neither interferes or obstructs the provider, or causes discomfort for the patient; and the flexible tube 12 may be routed in a way that doesn't disturb a dental dam placed in the patient's mouth. For example, the flexible tube 12 may be routed in a manner so that it parallels in inside of the patient's cheek before exiting the mouth of the patient. The first end 12A of the flexible tube 12 may be configured to be fluidly coupled to the delivery system for the anesthetic 18 such that the delivery system is capable of delivering the anesthetic 18 through the flexible tube 12, needle 14, and cannula 20 into the jaw bone of the dental patient one or more times.

When the second end 12B of the flexible tube 12 is fluidly coupled to the proximal end 20A of the cannula 20, the male friction lock 20C is inserted into the female friction lock 16A such that sufficient friction between them prevents the coupler 16 from becoming fluidly uncoupled from the proximal end 20A of the cannula 20 when the anesthetic 18 is delivered into the jaw bone, typically under a relatively low fluid pressure. However, this friction may be low enough so that the provider is still able to manually remove the coupler 16 from the proximal end 20A when desired. Other techniques of fluidly coupling the coupler 16 to the proximal end 20A of the cannula 20 may be used as well, including those known now in the art and those which may be developed in the future. The needle 14 may be construction of stainless steel, a metal alloy, or any other suitable material. As discussed above, the needle 14 may be designed so that it may be inserted into the hollow proximal end 20A of the cannula 20 when the cannula 20 and coupler 16 are fluidly coupled.

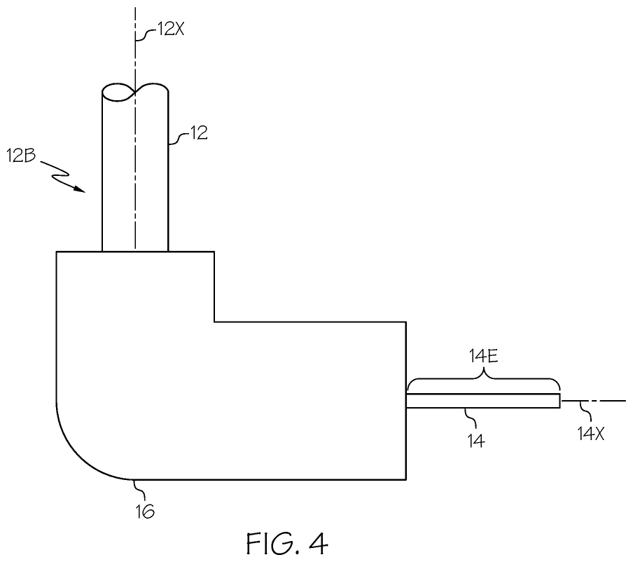

FIG. 4 shows the spatial relationship between the second end 12B of the flexible tube 12 and the needle 14. The second end 12B of the flexible tube 12 may have a longitudinal axis 12X. Likewise, the needle 14 may have a longitudinal axis 14X which may be substantially parallel to the portion of the needle 14 that may be inserted into the cannula. The needle 14 may be fluidly coupled to the second end 12B of the flexible tube 12 so that the needle 14 is at substantially a right angle with respect to the second end 12B of the flexible tube 12. That is, the longitudinal axis 12X of the second end 12B may be substantially at a right angle to the longitudinal axis 14X of the needle 14. Furthermore, a portion 14E of the needle 14 may extend beyond the end of the coupler 16. This portion 14E may be inserted into the proximal end of the cannula as discussed herein.

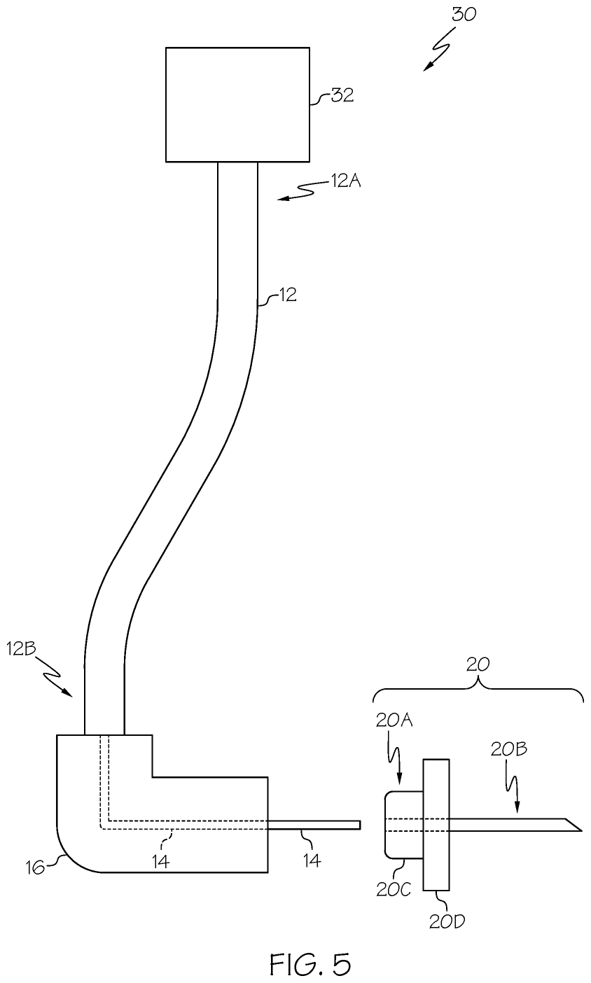

FIG. 5 illustrates another apparatus 30 for delivering an anesthetic into a jaw bone of a dental patient. The apparatus 30 may comprise a delivery system 32, a flexible tube 12 having a first end 12A and a second end 12B, a needle 14, a coupler 16, and a cannula 20 having a proximal end 20A and a distal end 20B. The first end 12A of the flexible tube 12 may be configured to be fluidly coupled to the delivery system 32, which may be capable of injecting the anesthetic into the flexible tube 12. The first end 12A of the flexible tube 12 may be fluidly coupled to the delivery system 32 by means of a Luer lock or other suitable connection. In other embodiments, the delivery system 32 may comprise an automatic delivery system which is likewise fluidly coupled to the flexible tube 12 at the first end 12A. The automatic delivery system may delivery the anesthetic into the flexible tube 12 in an automated manner, such as periodically or when commanded to do so by the dental provider via a button or switch electrically connected to the automatic delivery system, for example.

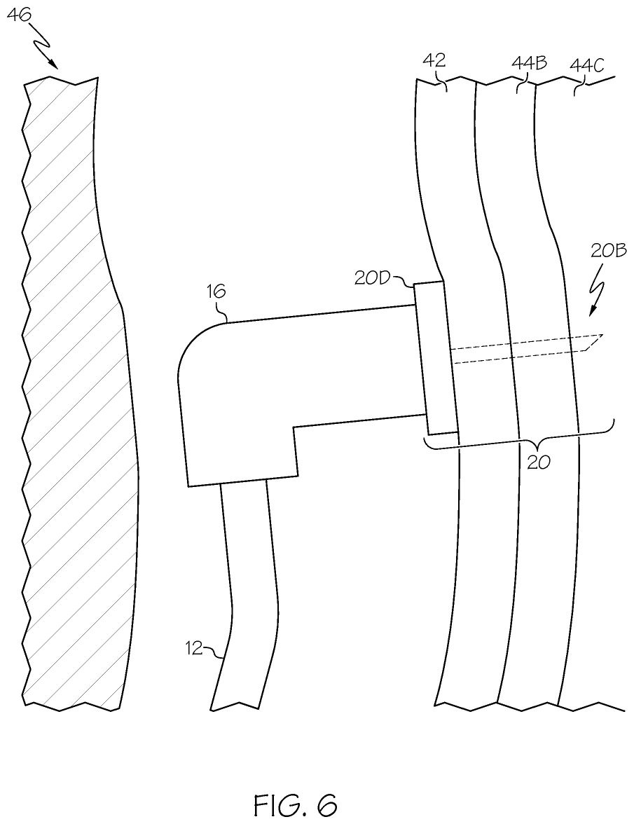

FIG. 6 shows a cross sectional view of a cannula 20 inserted into the jaw bone 44B, 44C of a dental patient according to one embodiment. The jaw bone 44B, 44C of the dental patient may be covered by an outermost layer of gingiva 42. The jaw bone 44B, 44C may comprise a layer of outer bone 44B, and cancellous bone 44C. As shown, the cannula 20 is inserted in a manner that the distal end 20B of the cannula 20 is disposed in the cancellous bone 44C. In this manner, the anesthetic is delivered from the delivery system into the cancellous bone 44C of the dental patient. The second end 12B of the flexible tube 12 may be fluidly coupled to the proximal end 20A of the cannula 20. This figure also depicts the cheek 46 of the dental patient. The flexible tube 12 may be configured in a manner so that it neither interferes nor obstructs the provider, nor causes discomfort for the patient. Furthermore, the flexible tube 12 may be routed in a way that does not disturb a dental dam that may be used during the procedure. Accordingly, the flexible tube 12 may be disposed at substantially a right angle with respect to the cannula 20. In this embodiment, the second end 12B of the flexible tube 12 may be configured to have a substantially right angle so that the protrusion of the proximal end 20A of the cannula 20, the second end 12B of the flexible tube 12, and the flexible tube 12 itself is minimized, which may reduce discomfort for the patient.

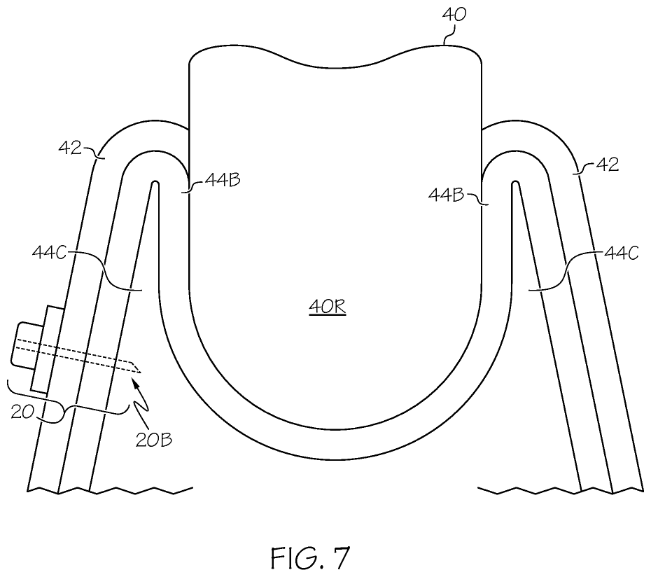

FIG. 7 depicts a cannula 20 inserted into the jaw bone 44B, 44C of a dental patient. Although a lower jaw bone is shown, it is contemplated that the same techniques may be used on the upper jaw bone. As discussed above, the jaw bone 44B, 44C may comprise a layer of outer bone 44B and cancellous bone 44C, and the layer of outer bone 44B may be covered by a layer of gingiva 42. The cannula 20 may be inserted through the gingiva 42 and the outer bone 44B of the dental patient so that it accesses the cancellous bone 44C near the tooth 40 subject to the dental procedure. The anesthetic may be typically delivered to the cancellous bone 44C of the dental patient in order to numb the patient quickly and effectively. The cannula 20 may be inserted so that its tip is near the root 40R of the tooth 40. Generally, the cannula 20 may be inserted at a substantially right angle to the jaw bone 44B, 44C of the patient. The cannula 20 may be inserted into the jaw bone 44B, 44C via any number of techniques which currently exist or may be developed in the future. As an example, the cannula 20 may be inserted using a dental drill with an X-Tip® Drill and Guide Sleeve. For this technique, the X-Tip® is drilled directly into the patient's jaw bone 44B, 44C; once the proper depth is reached, the drill portion is removed, leaving behind the cannula 20. As discussed above, it is expected that other techniques for inserting a cannula 20 into the cancellous bone 44C of the dental patient may be used as well.

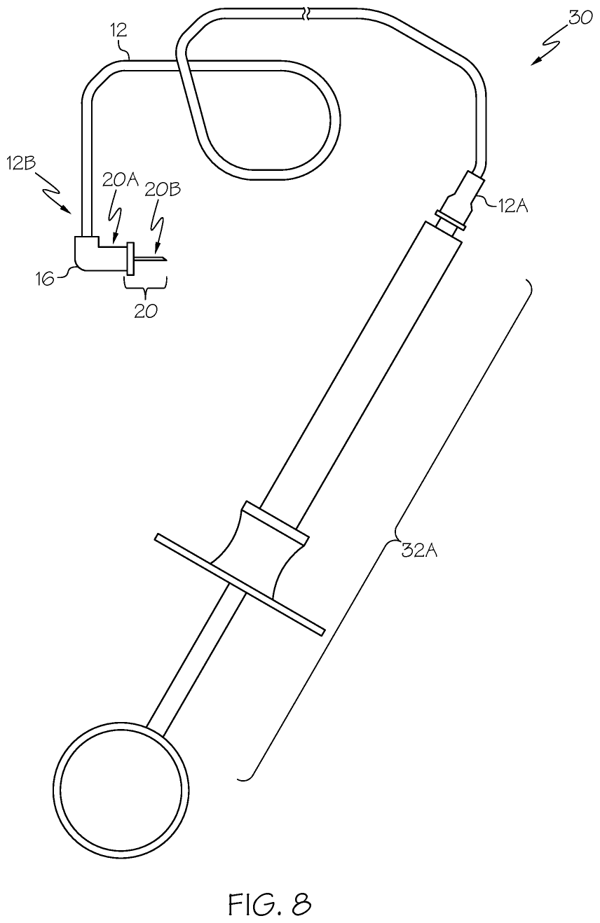

FIG. 8 illustrates an apparatus 30 for delivering an anesthetic into a jaw bone of a dental patient. The apparatus 30 may comprise a delivery system 32A for the anesthetic, a flexible tube 12 having a first end 12A and a second end 12B, a needle, a coupler 16, and a cannula 20 having a proximal end 20A and a distal end 20B. The first end 12A of the flexible tube 12 may be configured to be fluidly coupled to the delivery system 32A for anesthetic. In this figure, the delivery system 32A may comprise a syringe capable of injecting the anesthetic into the flexible tube 12. The medical provider may depress the plunger of the syringe in order to deliver the anesthetic to the dental patient. The first end 12A of the flexible tube 12 may be fluidly coupled to the delivery system 32A by means of a Luer lock or other suitable connection. In other embodiments, the delivery system may comprise an automatic delivery system which is likewise fluidly coupled to the flexible tube 12 at the first end 12A. The automatic delivery system may deliver the anesthetic into the flexible tube 12 in an automated manner, such as periodically or when commanded to do so by the dental provider.

Continuing to refer to FIG. 8, the second end 12B of the flexible tube 12 may be fluidly coupled to the needle such that the needle is at substantially a right angle to the second end 12B of the flexible tube 12. In this figure, the needle is already inserted into the proximal end 20A of the cannula 20 and is capable of delivering the anesthetic from the flexible tube 12 into the cannula 20. The coupler 16 may be disposed at the second 12B end of the flexible tube 12, may surround a portion of the needle, and may be configured to be removably affixed to the proximal end 20A of the cannula 20. When the coupler 16 is affixed to the proximal end 20A of the cannula 20, the coupler 16 may be configured to fluidly seal the second end 12B of the flexible tube 12 to the cannula 20. When the distal end 20B of the cannula 20 is inserted into the jaw bone of the dental patient, the needle is inserted into the proximal end 20A of the cannula 20, the coupler 16 is affixed to the proximal end 20A of the cannula 20, and the delivery system 32A is fluidly coupled to the first end 12A of the flexible tube 12, the delivery system 32A is capable of delivering the anesthetic through the flexible tube 12, the needle, and the cannula 20 into the jaw bone of the dental patient one or more times.

Other techniques for fluidly coupling the first end 12A of the flexible tube 12 to the delivery system 32A are also contemplated. For example, a self-tapping screw may be used which may screw into the delivery system 32A. The self-tapping screw may have a thin needle contained within it in order to puncture the membrane of a cartridge containing the anesthetic. The membrane and cartridge may be part of the delivery system 32A for the anesthetic, and this arrangement may allow the dental provider to easily and quickly fluidly couple the first end 12A of the flexible tube 12 to the delivery system 32A for the anesthetic.

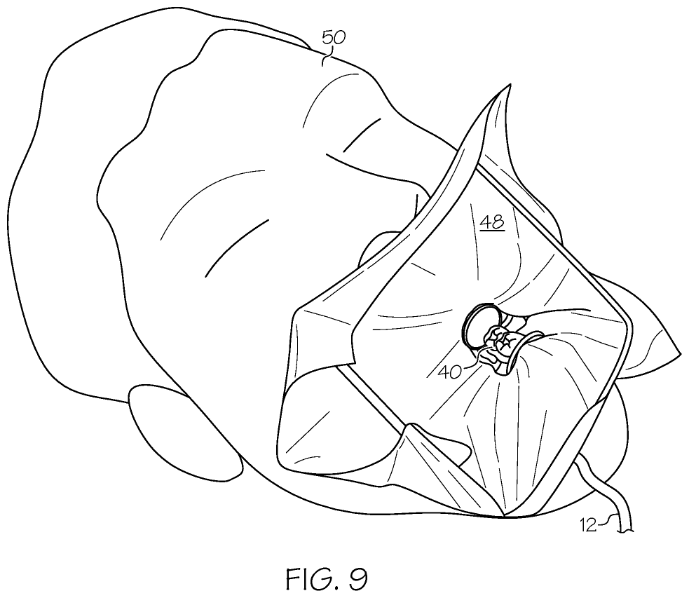

FIG. 9 depicts a dental dam 48 installed on a dental patient 50 having a dental procedure. The dental dam 48 may be placed over the tooth (or teeth) 40 subject to the procedure. As discussed above, the dental dam 48 may isolate the tooth 40 and may prevent germs from the patient's saliva from contaminating the tooth 40. This may be particularly important for dental procedures such as a root canal in which the internal components of the root must be disinfected before sealing. The apparatuses shown and described herein are capable of delivering the anesthetic to the dental patient 50 one or more times with a dental dam 48 in place. The flexible tube 12 of the apparatus may be routed from the cannula to the delivery system for anesthetic in a manner that does not interfere with the operation of the dental dam 48. Thus, the apparatuses may be capable of delivering the anesthetic to a dental patient 50 one or more times without the provider having to remove and reinstall the dental dam 48.

In addition to the apparatuses shown and described herein, methods are also disclosed for anesthetizing dental patients. In one embodiment, a method is disclosed for delivering an anesthetic to a dental patient, the method comprising: fluidly coupling a delivery system for the anesthetic to a first end of a flexible tube; inserting a distal end of a cannula into the jaw bone of the dental patient in a manner that allows the dental patient to be anesthetized; fluidly coupling a second end of a flexible tube onto a distal end of the cannula, wherein the second end of the flexible tube is configured to be at a substantially right angle to the cannula; and delivering the anesthetic from the delivery system through the flexible tube, the needle, and the cannula, and into the jaw bone of the dental patient one or more times.

While particular embodiments and aspects of the present invention have been illustrated and described herein, various other changes and modifications may be made without departing from the spirit and scope of the invention. Moreover, although various inventive aspects have been described herein, such aspects need not be utilized in combination. It is therefore intended that the appended claims cover all such changes and modifications that are within the scope of this invention.

What is claimed is:

1. An apparatus for delivering an anesthetic into a jaw bone of a dental patient, the apparatus comprising a flexible tube having a first end and a second end, a needle, and a coupler, wherein:
the first end of the flexible tube is configured to be fluidly coupled to a delivery system capable of injecting the anesthetic into the flexible tube;
the second end of the flexible tube is fluidly coupled to the needle such that the needle is at substantially a right angle to the second end of the flexible tube;
the needle is configured to be inserted into a proximal end of a cannula and, when inserted, is capable of delivering the anesthetic from the flexible tube into the cannula;
the coupler is disposed at the second end of the flexible tube, surrounds a portion of the needle, and is configured to be removably affixed to the proximal end of the cannula, wherein: when the coupler is affixed to the proximal end of the cannula, the coupler is configured to fluidly seal the second end of the flexible tube to the cannula; and
when a distal end of the cannula is inserted into the jaw bone of the dental patient, the needle is inserted into the proximal end of the cannula, the coupler is affixed to the proximal end of the cannula, and the delivery system is fluidly coupled to the first end of the flexible tube, the delivery system is capable of delivering the anesthetic through the flexible tube, the needle, and the cannula into the jaw bone of the dental patient one or more times.

2. The apparatus of claim 1, wherein the first end of the flexible tube comprises a Luer lock configured to be fluidly coupled to the delivery system.

3. The apparatus of claim 1, wherein the first end of the flexible tube comprises a self-tapping screw system configured to be fluidly coupled to the delivery system.

4. The apparatus of claim 1, wherein the delivery system is either a syringe or an automatic delivery system.

5. The apparatus of claim 1, wherein the coupler comprises a Luer lock.

6. The apparatus of claim 1, wherein the coupler comprises a female friction lock.

7. The apparatus of claim 1, wherein, when a dental dam is placed in the mouth of the dental patient, the apparatus is configured to deliver the anesthetic one or more times without removing the dental dam.

8. An apparatus for delivering an anesthetic into a jaw bone of a dental patient, the apparatus comprising a delivery system, a flexible tube having a first end and a second end, a needle, a coupler, and a cannula having a proximal end and a distal end, wherein:
the first end of the flexible tube is configured to be fluidly coupled to the delivery system capable of injecting the anesthetic into the flexible tube;
the second end of the flexible tube is fluidly coupled to the needle such that the needle is at substantially a right angle to the second end of the flexible tube;
the needle is configured to be inserted into the proximal end of the cannula and, when inserted, is capable of delivering the anesthetic from the flexible tube into the cannula;
the coupler is disposed at the second end of the flexible tube, surrounds a portion of the needle, and is configured to be removably affixed to the proximal end of the cannula, wherein: when the coupler is affixed to the proximal end of the cannula, the coupler is configured to fluidly seal the second end of the flexible tube to the cannula; and
when the distal end of the cannula is inserted into the jaw bone of the dental patient, the needle is inserted into the proximal end of the cannula, the coupler is affixed to the proximal end of the cannula, and the delivery system is fluidly coupled to the first end of the flexible tube, the delivery system is capable of delivering the anesthetic through the flexible tube, the needle, and the cannula into the jaw bone of the dental patient one or more times.

9. The apparatus of claim 8, wherein the first end of the flexible tube comprises a Luer lock configured to be fluidly coupled to the delivery system.

10. The apparatus of claim 8, wherein the first end of the flexible tube comprises a self-tapping screw system configured to be fluidly coupled to the delivery system.

11. The apparatus of claim 8, wherein the delivery system comprises either a syringe or an automatic delivery system.

12. The apparatus of claim 8, wherein the coupler comprises a Luer lock.

13. The apparatus of claim 8, wherein, when a dental dam is placed in the mouth of the dental patient, the apparatus is configured to deliver the anesthetic one or more times without removing the dental dam.

14. The apparatus of claim 8, wherein the coupler comprises a female friction lock.

15. A method for delivering an anesthetic into a jaw bone of a dental patient, the method comprising:
fluidly coupling a delivery system to a first end of a flexible tube;
inserting a distal end of a cannula into the jaw bone of the dental patient in a manner that allows the dental patient to be anesthetized;
fluidly coupling a second end of a flexible tube onto a proximal end of the cannula, wherein the second end of the flexible tube is configured to be at a substantially right angle to the cannula; and
delivering the anesthetic from the delivery system through the flexible tube, the needle, and the cannula, and into the jaw bone of the dental patient one or more times.

16. The method of claim 15, wherein the first end of the flexible tube comprises a Luer lock.

17. The method of claim 15, wherein the first end of the flexible tube comprises self-tapping screw system.

18. The method of claim 15, wherein the delivery system is either a syringe or an automatic delivery system.

19. The method of claim 15, wherein the second end of the flexible tube comprises a Luer lock.

20. The method of claim 15, wherein the method further comprises placing a dental dam in the mouth of the patient.
